(19) United States
(12) Patent Application Publication
(10) Pub. No.: US 2021/0196131 A1
(43) Pub. Date: Jul. 1, 2021

(54) MOBILE DEVICE-BASED CONGESTION PREDICTION FOR REDUCING HEART FAILURE HOSPITALIZATIONS

(71) Applicants: Board of Trustees of Michigan State University, East Lansing, MI (US); THE REGENTS OF THE UNIVERSITY OF MICHIGAN, Ann Arbor, MI (US)

(72) Inventors: Ramakrishna MUKKAMALA, Okemos, MI (US); Keerthana NATARAJAN, East Lansing, MI (US); Mohammed SAEED, Ann Arbor, MI (US)

(73) Assignees: Board of Trustees of Michigan State University, East Lansing, MI (US); THE REGENTS OF THE UNIVERSITY OF MICHIGAN, Ann Arbor, MI (US)

(21) Appl. No.: 17/057,990

(22) PCT Filed: May 24, 2019
(86) PCT No.: PCT/US2019/033934

§ 371 Date: Nov. 23, 2020

Related U.S. Application Data
(60) Provisional application No. 62/676,618, filed on May 25, 2018.

(51) Int. Cl. A61B 5/0205; A61B 5/00; A61B 5/318; A61B 5/024; A61B 5/08

(52) U.S. Cl. CPC A61B 5/0205; A61B 5/7275; A61B 5/318; A61B 5/02416; A61B 2562/0271; A61B 5/0816; A61B 2562/0219; A61B 2560/0223; A61B 5/681

(57) ABSTRACT

A method is presented for predicting onset of pulmonary congestion symptoms. The overall idea is to track rising left ventricular filling pressure (LVFP) in heart failure patients by exploiting significant changes in a pulsatile arterial waveform obtained with a mobile device and thereby avert hospitalizations. The stimulus for the changes may be metronomic deep breathing performed by the patient or a natural occurring arrhythmia such as atrial fibrillation or premature beats. For either stimulus, the extent of the amplitude variations depends on where the patient is on the Starling curve. If the patient is on the steep part of the curve, the variations will be large and LVFP will be low. If the patient is on the flatter part of the curve, the variations will be smaller and LFVP will be higher. These variations can be normalized in various ways to arrive at a congestion prediction index (CPI). When the CPI is below some threshold or is declining over time within a patient, then the patient may be on the verge of congestion symptoms.

### CROSS-REFERENCE TO RELATED APPLICATIONS

This application claims the benefit of U.S. Provisional Application No. 62/676,618 filed on May 25, 2018. The entire disclosure of the above application is incorporated herein by reference.

### FIELD

The present disclosure relates to systems and methods for mobile-device based congestion prediction for reducing heart failure hospitalizations.

### BACKGROUND

Frequent heart failure admissions significantly reduce the quality of patient life. A major cause of heart failure admissions is pulmonary congestion, which is defined as fluid accumulation in the tissue and air spaces of the lungs. In order to monitor pulmonary congestion, a patient may monitor his or her body weight. As an example, a rapid weight gain in a short period of time may be symptomatic of pulmonary congestion. However, a rapid weight gain often occurs after the accumulation of fluid in the tissues and air spaces of the lungs. As such, a rapid weight gain may be a late indicator of pulmonary congestion, and patients may need to be admitted to the hospital or seek other remedial measures prior to the rapid weight gain.

This section provides background information related to the present disclosure and is not necessarily prior art.

### SUMMARY

This section provides a general summary of the disclosure, and is not a comprehensive disclosure of its full scope or all of its features.

The overall idea is to track rising left ventricular filling pressure (LVFP)—a cardinal, early indicator of pulmonary congestion—in heart failure patients by exploiting significant changes in a pulsatile arterial waveform obtained with a mobile device and thereby avert hospitalizations. The pulsatile arterial waveform may be measured with a photo-plethysmography (PPG) sensor, which is already included in many mobile devices. The stimulus for the changes may be metronomic deep breathing performed by the patient (e.g., once a day) or a common, natural occurring arrhythmia such as atrial fibrillation or premature beats. Deep breathing causes pulse amplitude changes by varying venous return to the ventricle, whereas arrhythmias cause pulse amplitude changes by varying ventricular filling time. For either stimulus, the extent of the amplitude variations depends on where the patient is on the Starling curve. If the patient is on the steep part of the curve, the variations will be large and LVFP will be low. If the patient is on the flatter part of the curve, the variations will be smaller and LFVP will be higher. These variations can be normalized in various ways including by a respiratory tidal volume also measured with the mobile device (e.g., via the video camera or ECG electrodes) or the magnitude of the arrhythmia-induced pulse length changes to arrive at a congestion prediction index (CPI). If the patient has an intermittent arrhythmia, then the pulsatile arterial waveform can be monitored continuously to first detect the arrhythmia via large pulse length variations and then compute the CPI. When the CPI is below some threshold (set for a population of patients) or is declining over time within a patient, then the patient may be on the verge of congestion symptoms. This information can be relayed to the cardiologist who can then adjust patient diuretic therapy to avoid a hospitalization.

In one aspect, a method is presented for predicting an onset of pulmonary congestion symptoms using metronomic deep breathing as stimulus for changes in a pulsatile arterial waveform of a patient. The method includes: measuring, by a first sensor integrated into the mobile device, a pulsatile arterial signal from the patient during metronomic deep breathing; measuring, using a second sensor integrated into the mobile device, a respiratory signal from the patient during metronomic deep breathing; receiving, by a processor of the mobile device, the pulsatile arterial signal from the first sensor and the respiratory signal from the second sensor; determining, by the processor of the mobile device, magnitude of the amplitude variation of the pulsatile arterial signal and magnitude of the respiratory signal; and computing, by the processor of the mobile device, a congestion prediction index based on the magnitude of the amplitude variation of the pulsatile arterial signal and the magnitude of the respiratory signal.

The patient may be guided in performing metronomic deep breathing using cues issued by the mobile device. The cues issued by the mobile device may include auditory cues or visual cues that are configured to indicate a time to initiate each breath during metronomic deep breathing.

The magnitude of the amplitude variation of the pulsatile arterial signal may be determined by determining a difference between a maximum peak-to-peak amplitude of the pulsatile arterial signal and a minimum peak-to-peak amplitude of the pulsatile arterial signal over a respiratory cycle; determining a mean value based on the maximum peak-to-peak amplitude of the pulsatile arterial signal and the minimum peak-to-peak amplitude of the pulsatile arterial signal; and dividing the difference by the mean value.

In some embodiments, the magnitude of the respiratory signal is an average peak-to-peak amplitude of the respiratory signal. The congestion prediction index is the magnitude of the amplitude variation of the pulsatile arterial signal divided by the magnitude of the respiratory signal.

The method may further include comparing the congestion prediction index or changes in the congestion prediction index over time to a threshold and generating an alert in response the congestion prediction index being less than the threshold.

In another aspect, a method is presented for predicting an onset of pulmonary congestion symptoms using persistent arrhythmia (e.g., atrial fibrillation) as stimulus for changes in a pulsatile arterial waveform of a patient. The method includes: measuring, by a sensor, a pulsatile arterial signal from a patient with a persistent arrhythmia; receiving, by a processor of a computing device, the pulsatile arterial signal from the sensor; detecting, by the processor of the computing device, amplitude in the pulsatile arterial signal and length of the beats in the pulsatile arterial signal; and computing, by the processor of the computing device, a congestion prediction index based on the variation in the detected amplitudes and beat lengths.

In some embodiments, the congestion prediction index is computed as the slope of the line that relates the peak-to-peak amplitudes of the pulsatile arterial signal, normalized by mean peak-to-peak amplitude of the pulsatile arterial signal, to previous beat lengths of the pulsatile arterial signal. The previous beat lengths may also be normalized by mean beat length.

In other embodiments, the congestion prediction index is computed as standard deviation of the peak-to-peak amplitudes of the pulsatile arterial signal, normalized by the mean peak-to-peak amplitude of the pulsatile arterial signal, divided by the standard deviation of the beat lengths of the pulsatile arterial signal, normalized by mean beat length.

The method may further include comparing the congestion prediction index or changes in the congestion prediction index over time to a threshold and generating an alert in response to the congestion prediction index being less than the threshold.

In yet another aspect, a method is presented for predicting an onset of pulmonary congestion symptoms using an intermittent arrhythmia (e.g., paroxysmal atrial fibrillation or a premature beat) as stimulus for changes in a pulsatile arterial waveform of a patient. The method includes: measuring, by a sensor integrated into a wearable computing device, a pulsatile arterial signal from the patient; receiving, by a computer processor integrated into the wearable computing device, the pulsatile arterial signal from the sensor; analyzing, by the computer processor, the pulsatile arterial signal to detect an occurrence of an arrhythmia; and computing, by the computer processor, a congestion prediction index based on the amplitude variation of the pulsatile arterial signal during the arrhythmia.

In some embodiments, the method further includes detecting a period of reduced motion by the patient using an accelerometer integrated into the wearable computing device and analyzing the pulsatile arterial signal during the period of reduced motion.

In some embodiments, the method further includes detecting a period of non-vasoconstriction using a temperature sensor integrated into the wearable computing device and analyzing the pulsatile arterial signal during the period of non-vasoconstriction.

Detecting an occurrence of an arrhythmia can be based on variation in the beat length of the pulsatile arterial signal.

In some embodiments, the congestion prediction index is computed as slope of a line that relates peak-to-peak amplitudes of the pulsatile arterial signal, normalized by mean peak-to-peak amplitude of the pulsatile arterial signal, to previous beat lengths of the pulsatile arterial signal, normalized by mean beat length.

In other embodiments, the pulsatile arterial signal is analyzed to detect premature beat patterns that are similar in beat lengths and beat amplitude, and the congestion prediction index is computed as peak-to-peak amplitude of the beat following a longest beat normalized by peak-to-peak amplitude of a normal beat.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagram illustrating an example embodiment of a pulmonary congestion prediction system according to the present disclosure.

FIG. 2 is a component block diagram illustrating an example embodiment of the pulmonary congestion prediction system according to the present disclosure.

FIG. 3 is a diagram of another example embodiment of the pulmonary congestion prediction system according to the present disclosure.

FIG. 4 is a diagram of another example embodiment of the pulmonary congestion prediction system according to the present disclosure.

FIG. 5 is a flowchart illustrating an example method for obtaining a congestion prediction index value according to the present disclosure.

FIG. 6 is an example user interface of a mobile device of a pulmonary congestion prediction system according to the present disclosure.

FIG. 7 is a diagram of another example embodiment of the pulmonary congestion prediction system according to the present disclosure.

FIG. 8 is a flowchart illustrating an example method for obtaining a congestion prediction index value using a pulmonary congestion prediction system according to the present disclosure.

FIG. 9 is another flowchart illustrating another example method for obtaining a congestion prediction index value using a pulmonary congestion prediction system according to the present disclosure.

FIG. 10 is another flowchart illustrating another example method for obtaining a congestion prediction index value using a pulmonary congestion prediction system according to the present disclosure.

FIG. 11 is an example user interface of a mobile device of a pulmonary congestion prediction system according to the present disclosure.

Corresponding reference numerals indicate corresponding parts throughout the several views of the drawings.

### DETAILED DESCRIPTION

Example embodiments will now be described more fully with reference to the accompanying drawings.

Techniques are disclosed for predicting an onset of pulmonary congestion in chronic heart failure patients using a mobile device. In order to predict the onset of pulmonary congestion, the mobile device may detect and analyze significant changes in a patient's pulsatile arterial waveform. In response to the significant changes in the patient's pulsatile arterial waveform indicating the onset of pulmonary congestion, healthcare providers may subsequently take remedial actions in an outpatient setting in order to prevent and/or treat the onset pulmonary congestion (e.g., increase a diuretic dosage) and thereby prevent hospitalizations.

A pulmonary congestion prediction system may exploit respiratory-induced changes in a pulsatile arterial waveform. In one embodiment, the pulmonary congestion prediction system may analyze changes in the pulsatile arterial waveform while the patient is performing a normal or metronomic deep breathing routine in order to predict the onset of pulmonary congestion.

In other aspects, the pulmonary congestion prediction system may not exploit respiratory-induced changes in the pulsatile arterial waveform in order to predict an onset of pulmonary congestion. As an example, the pulmonary congestion prediction system may analyze changes in the pulsatile arterial waveform during a patient's sustained or intermittent arrhythmia (e.g., atrial fibrillation, atrial flutter, premature atrial contractions, premature ventricular contractions, etc.) in order to predict the onset of pulmonary congestion.

In addition to the pulmonary congestion prediction system exploiting changes in the pulsatile arterial waveform while the patient is performing a deep breathing routine or during an arrhythmia, the pulmonary congestion prediction system may determine an onset of pulmonary congestion using heart sounds, urine output, body weight, and other conventional symptoms of pulmonary congestion.

Furthermore, the prediction system may exploit changes in an invasive or non-invasive blood pressure or blood volume waveform to analyze a fluid responsiveness in critically ill patients or a blood volume waveform to analyze an efficacy of diuretic therapy for heart failure patients.

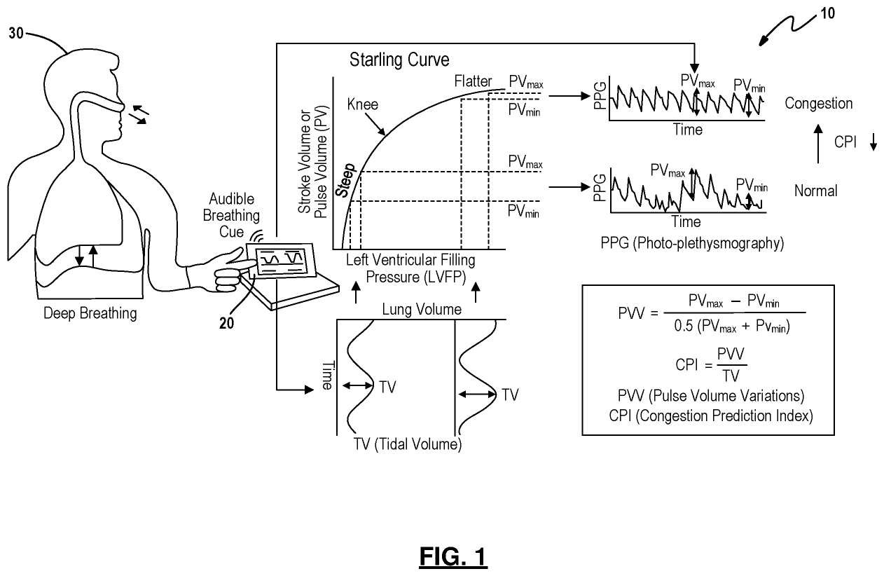

Referring now to FIG. 1, a diagram of an example embodiment of the pulmonary congestion prediction system 10 is shown. The pulmonary congestion prediction system 10 may be implemented by, for example, a mobile device 20 (e.g., a smartphone, smartwatch, etc.). In this embodiment, a patient 30 places an index finger on a sensing unit (e.g., a photoplethysmography sensor) of the mobile device 20 and simultaneously performs a normal deep breathing routine or a metronomic deep breathing routine, which may elicit a large respiratory input while fixing a respiratory rate of the patient 30. The mobile device 20 may provide visual or audio cues in order to ensure the patient 30 is properly performing the normal or metronomic deep breathing routine. As an example, the visual or audio cues may provide the patient 30 a time to begin each breath of the deep breathing routine. Subsequently, the mobile device 20 may generate, based on measurements received by the sensing unit and other sensors described below in further detail, a pulsatile arterial signal (e.g., a blood volume waveform, etc.) and a respiratory signal (e.g., a lung volume waveform) of the patient 30.

According to Starling's law, amplitude variations of the blood volume waveform may be associated with a left ventricular filling pressure (LVFP), which is a well-known early indicator of congestion. As described herein, a blood volume waveform is defined as a signal that is representative of a measured blood volume in an artery or capillary. As an example, in response to a large difference between a pulse volume maximum amplitude (PVₘₐₓ) and pulse volume minimum amplitude (PVₘᵢₙ) of a blood volume waveform over a respiratory cycle, the LVFP value is lower. Consequently, larger differences between the PVₘₐₓ and the PVₘᵢₙ of the blood volume waveform over a respiratory cycle are associated with a reduced likelihood of onset pulmonary congestion. Furthermore, in response to a small difference between the PVₘₐₓ and PVₘᵢₙ of the blood volume waveform over a respiratory cycle, the LVFP value is higher. As such, smaller differences between the PVₘₐₓ and PVₘᵢₙ are associated with an increased likelihood of onset pulmonary congestion.

Accordingly, the mobile device 20 may generate a pulse volume variation value (PVV) based on amplitude variations of the blood volume waveform over a respiratory cycle. As an example, the mobile device 20 may generate the PVV based on the PVₘₐₓ and PVₘᵢₙ of a respiratory cycle Specifically, the mobile device 20 may generate the PVV using the following formula:

$$P V V = \frac{P {V}_{ma x} - P {V}_{min}}{0.5 ( P {V}_{max} + P {V}_{min} )} \qquad ( 1 )$$

It should be understood to one of ordinary skill in the art that other characteristics of the blood volume waveform may be used to calculate the PVV, such as a standard deviation of the pulse volumes over a respiratory cycle, pulse volume average value of at least one cycle, a root-mean-square (RMS) pulse volume value of at least one cycle, etc.

Based on the PVV and the lung volume waveform, the mobile device 20 may generate a congestion prediction index (CPI), which is a value that indicates an onset of pulmonary congestion. As an example, the mobile device 20 may compute a tidal volume (TV) of the lung volume waveform, which is an average peak-to-peak amplitude of the lung volume waveform. Based on the PVV and the TV, the mobile device 20 may generate the CPI using the following formula:

$$CPI = \frac{P V V}{T V} \qquad ( 2 )$$

It should be understood to one of ordinary skill in the art that other characteristics of the lung volume waveform may be used to calculate the CPI, such as a maximum amplitude of the lung volume waveform, a minimum amplitude of the lung volume waveform, a standard deviation lung volume value, etc.

Based on the CPI, the mobile device 20 can predict an onset of pulmonary congestion. As an example, a lower CPI may indicate a higher likelihood of onset of pulmonary congestion, while a higher CPI may indicate a lower likelihood of onset of pulmonary congestion. In one embodiment, a CPI that is below a threshold CPI value may indicate an onset of pulmonary congestion. Accordingly, the patient 30 and/or mobile device 20 may alert a healthcare provider of the onset of pulmonary congestion if the CPI is below the threshold CPI value and, in response, the healthcare provider may take remedial actions to prevent a hospitalization and/or pulmonary congestion, such as increasing a diuretic dosage of the patient 30.

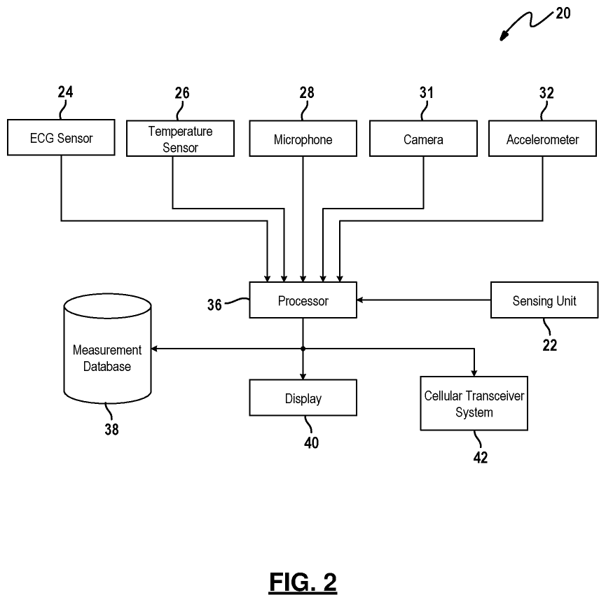

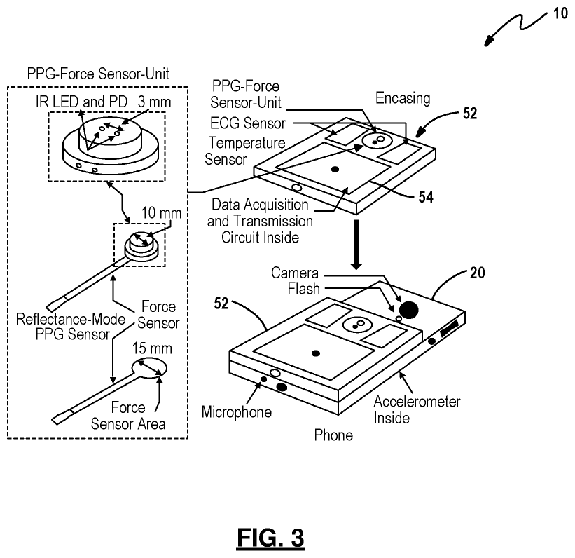

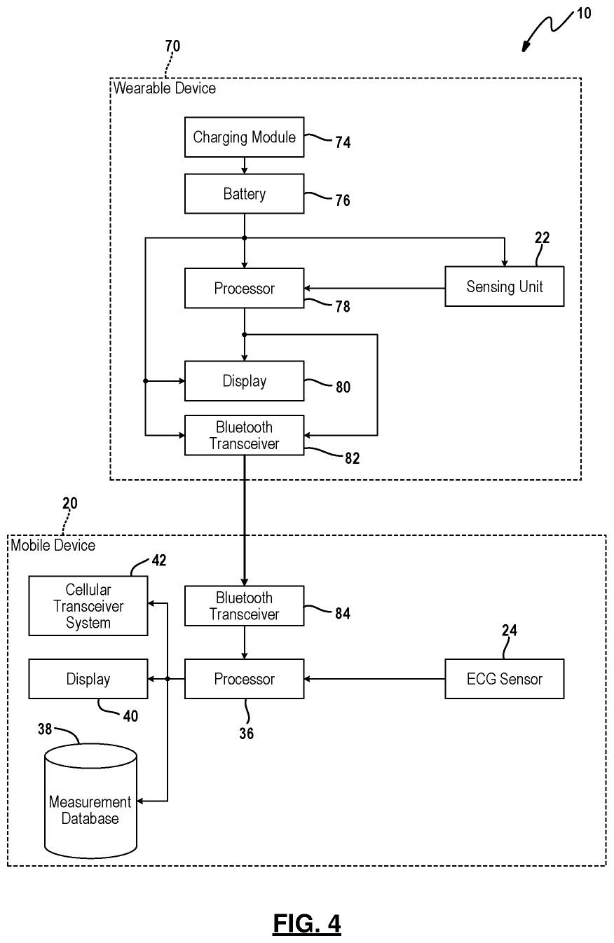

FIG. 2 illustrates a component block diagram of the mobile device 20. While this embodiment illustrates each of the components as part of the mobile device 20, some of the components may be implemented separately from the mobile device 20, as shown below in FIGS. 3-4. The mobile device 20 generally comprises a sensing unit 22, an electrocardiogram (ECG) sensor 24, a temperature sensor 26, a microphone 28, a camera system 31, an accelerometer 32, and a processor 36. Additionally, the mobile device 20 may include a measurement database 38, a display 40, and a cellular transceiver system 42. It should be understood by one of ordinary skill in the art that the mobile device 20 may include other components to carry out the functionality described herein.

In order to generate the blood volume waveform, the mobile device 20 may include a processor 36 that receives blood volume measurements from a sensing unit 22. In order to carry out the functionality described herein, the processor 36 may be configured to execute instructions stored in a nontransitory memory, such as a random-access memory (RAM) and/or read-only memory (ROM). As an example, the sensing unit 22 may include a reflectance-mode photoplethysmography (PPG) sensor (e.g., a pulse oximeter) that detects and measures blood volume oscillations and a pressure sensor that detects and measures an amount of applied pressure. Based on the measurements received from the sensing unit 22, the processor 36 is configured to generate the PVV and CPI.

In order to generate the lung volume waveform, the processor 36 may receive measurements from an electrocardiogram (ECG) sensor 24. As an example, the ECG sensor 24 may include a plurality of dry electrodes that detect R-wave amplitude variations associated with breathing-induced changes in thoracic impedance. Based on the R-wave amplitude variations, the processor 36 may generate the lung volume waveform.

In one variant, to generate the lung volume waveform, the processor 36 may receive measurements from a temperature sensor 26. As an example, the patient 30 may breathe with the temperature sensor 26 in front of his or her nose while keeping his or her mouth closed. Subsequently, the temperature sensor 26 may generate cyclical waveforms, and the processor 36 may generate the lung volume waveform based on the cyclical waveforms and a mean of the cyclical waveforms.

In another variant, to generate the lung volume waveform, the processor 36 may receive measurements from a microphone 28. As an example, the microphone 28 may be configured to obtain, at a sampling rate of 20 kHz, thoracic wall motion measurements that are generated in response to normal or metronomic deep breathing via a sonar principle. As such, the processor 36 may be configured to generate the lung volume waveform based on the thoracic wall motion measurements.

In yet another variant, to generate the lung volume waveform, the processor 36 may receive measurements from a camera system 31. As an example, a a camera 31 is directed at the chest, abdomen, face, neck, or upper body of the patient 30. In response to receiving image data from the camera representing the patient's body movement during normal or metronomic deep breathing, the processor 36 is configured to generate the lung volume waveform based on the video data.

Alternatively, to generate the lung volume waveform, the processor 36 may receive measurements from an accelerometer 32. As an example, the patient 30 may place the mobile device 20 on his or her chest or abdomen to measure thoracic wall motion caused by the normal or metronomic deep breathing. As such, the processor 36 may be configured to generate the lung volume waveform based on the thoracic wall motion measurements.

In response to generating the blood volume waveform and the lung volume waveform, the processor 36 may generate the PVV based on the blood volume waveform and the TV based on the lung volume waveform, as described above. Based on the PVV and the TV, the processor 36 may subsequently determine the CPI, as described above.

In response to generating the blood volume waveform, the lung volume waveform, and the CPI, the processor 36 may generate an entry that includes the patient identification information, time information, waveform characteristics, and flags corresponding to the transmission, receipt, and processing of the corresponding entry. Subsequently, the processor 36 may store the entry in a measurement database 38. An example data structure of the entry is provided below:

Entry No.: 000001
Patient ID:
Time of CPI Measurement:
CPI:
TV:
PVV:
PVₘₐₓ:
PVₘᵢₙ:
Provider Alert Transmission:
Provider Alert Received:
Provider Alert Processed:

Additionally, the processor 36 may provide a signal to the display 40 based on information of the entry. As an example, in response to receiving the signal, the display 40 may generate a graphic corresponding to the blood volume waveform and the lung volume waveform and/or text corresponding to the various characteristics of the waveforms, such as the CPI, TV, PVV, PVₘₐₓ, and PVₘᵢₙ. An example graphic and text of the display 40 corresponding to the waveforms and the characteristics of the waveforms, respectively, are shown below in FIG. 4.

Additionally, the processor 36 may provide the signal to the cellular transceiver system 42 of the mobile device 20. The cellular transceiver system 42 may be implemented by various filtering, amplifying, and frequency mixing circuits in order to transmit and receive telemetric signals. In response to receiving the signal, the cellular transceiver system 42 may be configured to transmit a message corresponding to the entry to a remote server (not shown). Accordingly, a healthcare provider of the patient 30 may remotely monitor the patient's CPI measurements and take any remedial measures, if necessary.

Moreover, the cellular transceiver system 42 may receive messages from the remote server indicating that the healthcare provider has received and/or processed the original transmitted message. As such, the processor 36 may update the flags of the corresponding entry in response to receiving the message indicating the healthcare provider has received and/or processed the original transmitted message.

Referring to FIG. 3, a diagram of another example embodiment of the pulmonary congestion prediction system 10 is shown. This embodiment is similar to the pulmonary congestion prediction system described in FIG. 2, but in this embodiment, the sensing unit 22, the ECG sensor 24, and the temperature sensor 26 are integrated within an encasing 52 as opposed to the mobile device 20. The encasing 52 is physically coupled to the mobile device 20. Furthermore, the encasing 52 includes a transmission circuit 54 that is configured to transmit the measurements obtained by the sensing unit 22, the ECG sensor 24, and the temperature sensor 26 to the mobile device 20. As an example, the transmission circuit 54 may be implemented by a Bluetooth transceiver system or other communication system suitable for communicating with the mobile device 20.

In FIG. 4, a component block diagram of another example embodiment of the pulmonary congestion prediction system 10 is shown. In this embodiment, the sensing unit 22 is integrated within a wearable device 70 (e.g., a smartwatch, a wristband, a ring, etc.), and the ECG sensor 24 is integrated within the mobile device 20. Alternatively, the ECG sensor 24 may be integrated within the wearable device 70, and the sensing unit 22 may be integrated within the mobile device 20. In other aspects, the sensing unit 22 and the ECG sensor 24 may be integrated within the wearable device 70.

The wearable device 70 generally includes a charging module 74 that connects a battery 76 to an external power supply, and the battery 76 provides power to a processor 78, a display 80, a Bluetooth transceiver 82, and the sensing unit 22. It should be understood by one of ordinary skill in the art that the wearable device 70 may include other components to carry out the functionality described herein.

The processor 78 receives blood volume measurements from the sensing unit 22. When the wearable device 70 is implemented by a wristband, the sensing unit 22 may include a reflectance-mode PPG sensor that detects and measures blood volume oscillations. Alternatively, when the wearable device 70 is implemented by a ring, the sensing unit 22 may include a transmission-mode PPG sensor that detects and measures blood volume oscillations.

In response to the processor 78 receiving the blood volume measurements from the sensing unit 22, the processor 78 is configured to transmit the blood volume measurements obtained by the sensing unit 22 to the mobile device 20 via Bluetooth transceivers 82, 84. Additionally or alternatively, the processor 78 may provide a signal to the display 80 with instructions to display text and/or graphics corresponding to the blood volume measurement characteristics and/or the visual cues described above. In order to carry out the functionality described above, the processor 78 is configured to execute instructions stored in a nontransitory memory, such as a random-access memory (RAM) and/or a read-only memory (ROM).

As described above, the ECG sensor 24 is configured to generate a lung volume measurements based on detected R-wave amplitude variations associated with breathing-induced changes in thoracic impedance. In other embodiments, the mobile device 20 may include the temperature sensor 26, the microphone 28, the camera system 31, or the accelerometer 32 in order to generate the lung volume measurements.

In response to the mobile device 20 receiving the blood volume measurements from the wearable device 70 and the lung volume measurements from the ECG sensor 24, the processor 36 may generate a blood volume waveform and lung volume waveform. Based on the blood volume waveform and the lung volume waveform, the processor 36 may determine the PVV, TV, and CPI, as described above.

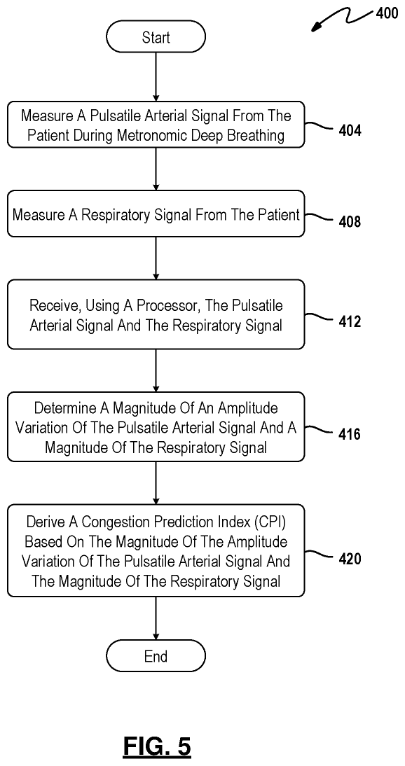

Referring to FIG. 5, a flowchart 500 describing an example method for obtaining a congestion prediction index value using the pulmonary congestion prediction system is shown. This method may be executed by the processor 36 in response to receiving blood volume and lung volume measurements from at least one of the sensors described above in FIG. 2. The method begins at 504, where the sensing unit 22 measures a pulsatile arterial signal (e.g., a blood volume waveform) from the patient during a metronomic deep breathing routine. At 508, at least one of the ECG sensor 24, temperature sensor 26, microphone 28, camera system 31, and accelerometer 32 measures a respiratory signal (e.g., a lung volume waveform) from the patient. At 512, the processor 36 receives the pulsatile arterial signal and the respiratory signal from the corresponding sensor. At 516, the processor 36 determines a magnitude of amplitude variation of the pulsatile arterial signal over a respiratory cycle (e.g., PVₘᵢₙ and PVₘₐₓ and/or PVV) and a magnitude of the respiratory signal (e.g., TV). At 520, the processor 36 derives the CPI based on the magnitude of amplitude variation of the pulsatile arterial signal and the magnitude of the respiratory signal.

Because the respiratory signal is a proportional measurement, it may need to be calibrated to yield a milliliter value. The mobile device will also allow for calibrating the TV to units of ml. For example, the patient could breathe into a Spirobag; the respiratory waveform can then be extracted with the mobile device; and a calibration factor can finally be determined as the ratio of the known Spirobag volume to the waveform amplitude. This calibration factor could be obtained during a one-time visit to the cardiologist and used thereafter to measure absolute TV. The calibrated TV measurement may not be accurate enough for inter-patient comparisons. However, the CPI is useful for tracking changes within a patient with or without calibration.

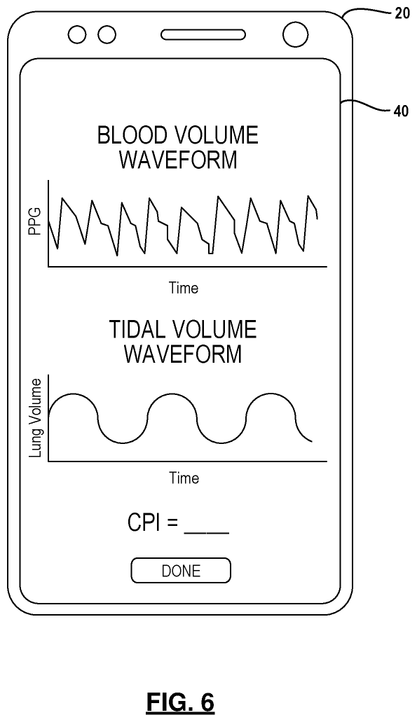

FIG. 6 illustrates an example user interface of display 40 of the mobile device 20 in response to the processor 36 generating an entry. As shown in FIG. 6, in response to receiving the signal from the processor 36, the display 40 generates a graphic of the blood volume waveform and the lung volume waveform and text corresponding to the CPI. The application may also assess artifact due to motion (e.g., based on a variable contact pressure) and the presence of arrhythmia (based on a highly variable pulse length). The application could display either CPI as the ratio of PVV to TV for example or “try again” if artifact or arrhythmia is detected. The application may also come with a video tutorial to explain how to use the device.

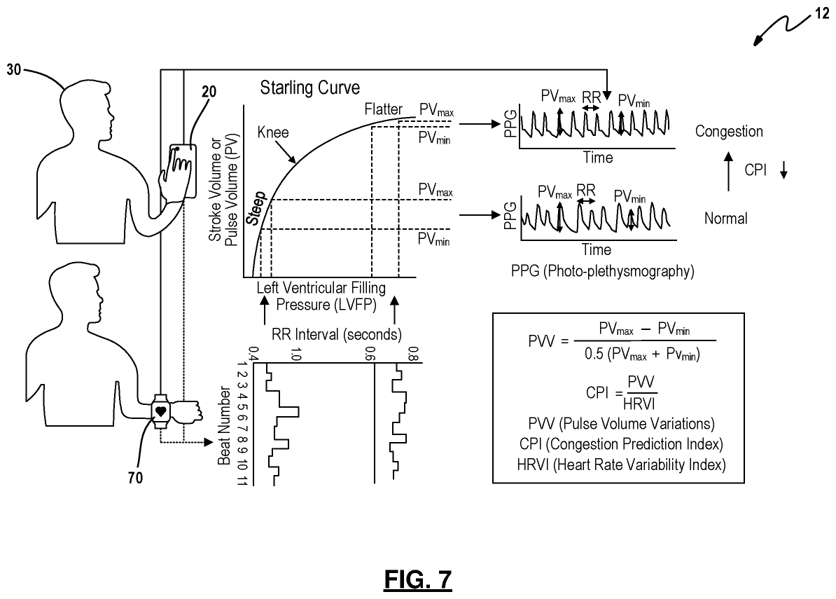

The embodiments described above in FIGS. 1-6 discuss pulmonary congestion prediction systems 10 that analyze changes in the pulsatile arterial waveform while the patient 30 is performing a normal or metronomic deep breathing routine in order to predict the onset of pulmonary congestion. The embodiments discussed below in FIGS. 7-11 are similar to the pulmonary congestion prediction systems 10, but in the below embodiments, the pulmonary congestion prediction system analyzes changes in the pulsatile arterial waveform during a patient's sustained or intermittent arrhythmia as opposed to a normal or metronomic deep breathing routine.

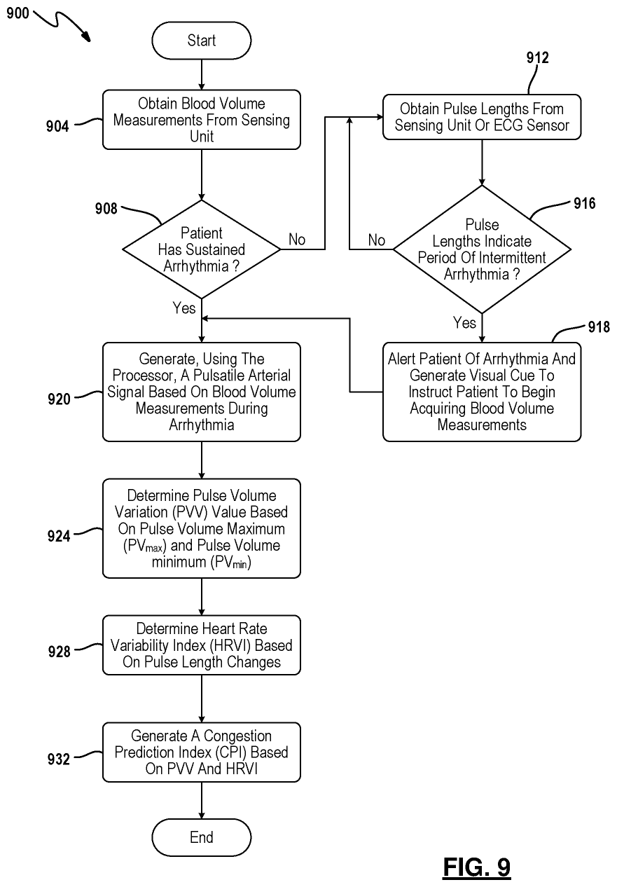

Now referring to FIG. 7, a diagram of an example embodiment of pulmonary congestion prediction system 12 is shown. In this embodiment, the patient 30 places an index finger on at least one sensor of the mobile device 20 during an arrhythmia (e.g., atrial fibrillation, atrial flutter, premature atrial contractions, premature ventricular contractions, etc.), and the mobile device 20 is configured to generate a pulsatile arterial signal (e.g., a blood volume waveform) based on data received from the sensing unit 22 (not shown), which may be implemented by a PPG sensor. Alternatively, the patient 30 may use a wearable device 70, such as a wristband, watch, or ring, to obtain the blood volume measurements and subsequently communicate the sensor data to the mobile device 20 or analyze the data on board the wearable device 70. Furthermore, the mobile device 20 and/or wearable device 70 may provide visual or audio cues in order to inform the patient 30 that he or she is experiencing an intermittent arrhythmia, as described below in further detail in FIG. 9.

The mobile device 20 may generate the PVV based on various characteristics of the blood volume waveform, such as amplitude variations of the blood volume waveform. As an example, the mobile device 20 may generate the PVV based on the PVₘₐₓ and PVₘᵢₙ of the blood volume waveform during a sampling period of atrial fibrillation. Specifically, the mobile device 20 may generate the PVV using the following formula:

$$P V V = \frac{P {V}_{max} - P {V}_{min}}{0.5 ( P {V}_{max} + P {V}_{min} )} \qquad ( 3 )$$

Based on the PVV and beat-to-beat pulse length changes of the blood volume waveform, the mobile device 20 may generate a heart rate variability index (HRVI) and the CPI. As an example, during the sampling period, the mobile device 20 may sum each pulse length (i.e., duration of each beat) and subsequently determine a mean and standard deviation of the pulse lengths. In some embodiments, the HRVI may be the standard deviation of the pulse lengths. Subsequently, the mobile device 20 may generate the CPI using the following formula:

$$CPI = \frac{P V V}{HRVI} \qquad ( 4 )$$

It should be understood by one of ordinary skill in the art that various metrics that are indicative of the pulse length changes of the blood volume waveform or indicative of heart rate variability may be used as the HRVI.

Based on the CPI, the mobile device 20 and/or wearable device 70 can predict an onset of pulmonary congestion. As an example, a lower CPI may indicate a higher likelihood of onset of pulmonary congestion, while a higher CPI may indicate a lower likelihood of onset of pulmonary congestion. In one embodiment, a CPI that is below a threshold CPI value may indicate an onset of pulmonary congestion. In another embodiment, changes in the CPI value over time is compared to a threshold. Accordingly, the patient 30 and/or mobile device 20 may alert a healthcare provider of the onset pulmonary congestion if the CPI is below the threshold CPI value and, as such, the healthcare provider may take remedial actions to prevent a hospitalization and/or pulmonary congestion, such as increasing a diuretic dosage of the patient 30.

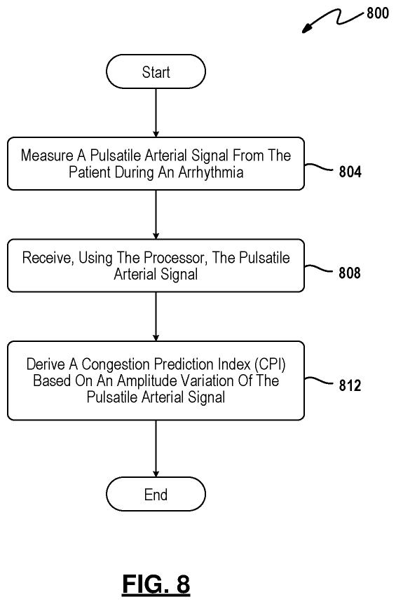

FIG. 8 is a high-level flowchart 800 describing an example method for obtaining the CPI using the pulmonary congestion prediction system 12. This method may be executed by the processor 36 in response to receiving blood volume measurements from the sensing unit 22. The method begins at 804, where one of the wearable device 70 and the mobile device 20 measures a pulsatile arterial signal from the patient 30 during an arrhythmia. At 808, the processor 36 receives the pulsatile arterial signal, and at 1012, the processor 36 derives the CPI based on an amplitude variation and possibly the pulse length variation of the pulsatile arterial signal.

Referring to FIG. 9, a detailed flowchart 900 describing an example method for obtaining the CPI during a sustained or intermittent arrhythmia using the pulmonary congestion prediction system 12 is shown. This method may be executed by the processor 36 in response to at least one of the mobile device 20 and the wearable device 70 being turned on. As an example, if the patient 30 suffers from intermittent arrhythmias, the processor 36 is configured to identify when the arrhythmia is occurring and generate CPI measurements only during periods of intermittent arrhythmia. If the patient 30 suffers from sustained arrhythmia, the processor 36 may be configured to continuously generate CPI measurements or generate CPI at the will of the patient. The method will now be described below in further detail.

At 904, the processor 36 obtains blood volume measurements from the sensing unit 22. At 908, the processor 36 determines whether the patient 30 has sustained arrhythmia. This determination may be based on, for example, a user profile associated with the patient 30 or a value inputted by the patient 30 on the mobile device 20 indicating that the patient 30 suffers from sustained arrhythmia. If the patient 30 has sustained arrhythmia, the method proceeds to 920; otherwise, the method proceeds to 912. At 912, the processor 36 analyzes the pulsatile arterial signal to detect an occurrence of an arrhythmia. The system can detect the occurrence of an arrhythmia by analyzing beat lengths in the pulsatile arterial signal. The occurrence of an arrhythmia may be determined from the same sensing unit 22 that measured the pulsatile arterial signal or from a different sensor, for example a signal from an electrocardiogram sensor. In some embodiments, a period of reduced motion by the patient is detected using an accelerometer integrated into the wearable computing device and the pulsatile arterial signal is analyzed only during the period of reduced motion to detect an arrhythmia. In other embodiments, a period of non-vasoconstriction is detected using a temperature sensor integrated into the wearable computing device and the pulsatile arterial signal is analyzed only when the temperature measurements are within a predefined range (i.e., indicating non-vasoconstriction periods).

At 916, the processor 36 determines whether the pulse lengths indicate that the patient 30 is experiencing a period of intermittent arrhythmia. As an example, the processor 36 may determine that the patient is experiencing a period of intermittent arrhythmia in response to a variation of the pulse lengths, such as a standard deviation or variance, exceeding a predetermined threshold. As another example, the system detects premature beat patterns (i.e., short beat followed by a long beat) as an indicator for the occurrence of an intermittent arrhythmia. If the pulse lengths indicate that the patient 30 is experiencing a period of intermittent arrhythmia, the method proceeds to 918; otherwise, the method proceeds to 912. At 918, the processor 36 generates a signal that alerts the patient 30 of the intermittent arrhythmia and that causes the display 40 to generate a visual cue that instructs the patient 30 to begin acquiring blood volume measurements. In other embodiments, the blood volume measurement is obtained automatically without patient knowledge, for example by a wearable device.

At 920, the processor 36 generates a pulsatile arterial signal based on the blood volume measurements during the arrhythmia. At 924, the processor 36 determines the PVV based on the PVₘₐₓ and PVₘᵢₙ of the pulsatile arterial signal. At 928, the processor 36 determines the HRVI based on the pulse length changes, as described above. At 932, the processor 36 generates the CPI based on the PVV and the HRVI.

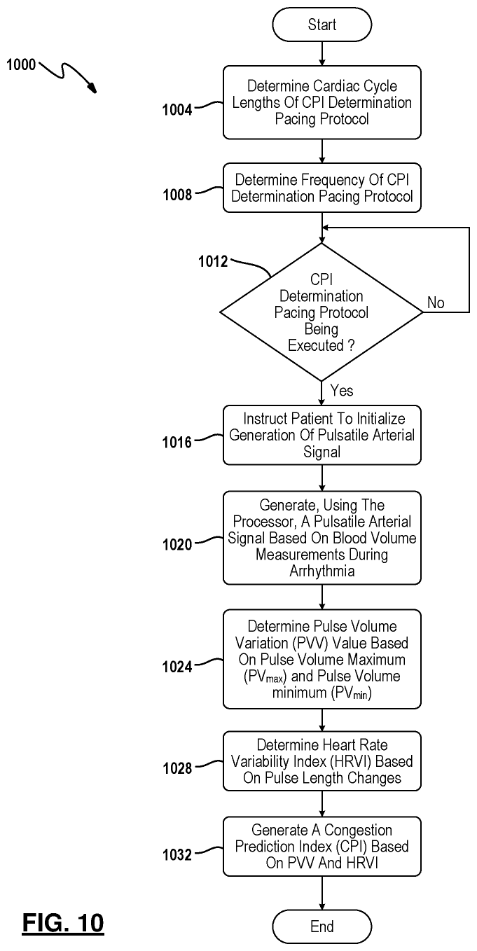

Referring to FIG. 10, another detailed flowchart 1000 describing an example method for obtaining the CPI during an intermittent arrhythmia using the pulmonary congestion prediction system 12 is shown. This method may be executed when, for example, the patient 30 has an implanted cardiac pacing device, such as a pacemaker, cardioverter, defibrillator or any other device that is configured to perform cardiac pacing. Specifically, the cardiac pacing device may be configured to a randomized pacing protocol in the patient 30, and as such, the mobile device 20 may be configured to determine the CPI during the pacing protocol.

At 1004, the cardiac cycle lengths of a CPI determination pacing protocol of the cardiac pacing device are determined. In fact, they are known. The cardiac cycle lengths may each have the same value or have different values, and the cardiac cycle lengths may be determined to mitigate a mechanical restitution of the heart and postextrasystolic potentiation effects. At 1008, the frequency of the CPI determination pacing protocol (e.g., daily, every other day, etc.) is determined by, for example, the healthcare provider. At 1012, the mobile device 20 determines whether the CPI determination pacing protocol is being executed. As an example, the mobile device 20 may communicate with the cardiac pacing device using a secure Bluetooth communication link in order to determine whether the CPI determination pacing protocol is being executed. If so, the method proceeds to 1016; otherwise, the method remains at 1012.

At 1016, the processor 36 generates a signal alerting the patient to initialize the generation of the pulsatile arterial signal by, for example, using a visual cue on the display 40. At 1020, the processor 36 generates a pulsatile arterial signal based on the blood volume measurements during the arrhythmia. At 1024, the processor 36 determines the PVV based on the PVₘₐₓ and PVₘᵢₙ of the pulsatile arterial signal. At 1028, the processor 36 determines the HRVI based on the pulse length changes, as described above. At 1032, the processor 36 generates the CPI based on the PVV and the HRVI.

The mobile device may compute an effective CPI during atrial fibrillation in several ways. Examples follow. The PVV can be quantified as shown in FIG. 7 or as the standard deviation of the pulse amplitudes. The PVV may be normalized by the extent of the pulse length variability, as quantified in terms of the standard deviation of the pulse lengths or otherwise. Alternatively, the slope of the line relating the pulse amplitudes to the previous pulse intervals may be determined. In computing the CPI, all of the beats may not necessarily be used. For example, short beats or short beats followed by long beats may be omitted, as these beats may reflect not only the Starling effect but also potentially changes in ventricular contractility (e.g., due to mechanical restitution or post-extrasystolic potentiation). In one embodiment, short beats are those beats whose beat length fall into the shortest 10% of all beat lengths. Likewise, long beats are those beats whose beat length fall into the longest 10% of all beat lengths. In addition, the CPI may be normalized by the mean pulse length in order to compare, for example, atrial fibrillation patients with and without rate control.

The application can include automatic algorithms to detect waveform artifact due to motion or otherwise. The application may output the CPI computed from the waveform or “try again” when non-trivial artifact is detected or some other algorithmic problem occurs.

The patient may have other electrical anomalies in addition to atrial fibrillation, such as atrio-ventricular block or aberrant conduction, and the CPI measurement may still be used for intra-patient (but perhaps not inter-patient) assessment.

For patients with sporadic premature beats, two approaches can be employed. One approach is to find premature beat patterns that are similar over time. For example, the time intervals of the premature beat and compensatory pause should be similar (i.e., beats within a predefined tolerance of each other (e.g., within 10%)) to equalize for potential ventricular contractility and filling time differences. In addition, the amplitude of the premature beat should be similar to equalize for other differences. Then, the pulse amplitude of the compensatory pause beat normalized by the pulse amplitude of a normal beat may be computed, for example, as the CPI. Another approach is to correct the pulse amplitude of the compensatory pause beat normalized by the pulse amplitude of a normal beat by the time intervals and/or amplitude of the premature beat. For example, such a correction could be implemented with multiple regression analysis.

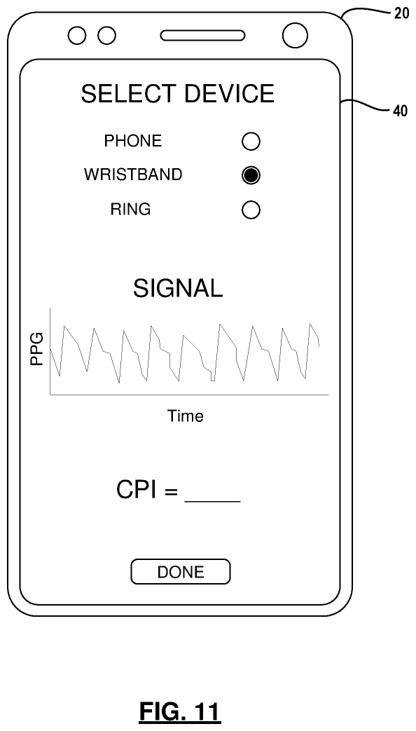

Referring to FIG. 11, an example user interface of the display 40 of the mobile device 20 is shown. As shown in FIG. 11, the display 40 generates the blood volume waveform and text corresponding to the CPI. Furthermore, the display 40 generates graphical user interface (GUI) elements that enable the patient 30 to select the type of device that is obtaining the blood volume measurements and/or heartbeat detection measurements. In response to selecting the GUI element associated with the wearable device 70 (i.e., the wristband or ring), the mobile device 20 may initiate a Bluetooth pairing function with the wearable device 70 in order to establish a secure communication link between the mobile device 20 and the wearable device 70.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A method for predicting an onset of pulmonary congestion symptoms in a patient using a mobile device, comprising:
measuring, by a first sensor integrated into the mobile device, a pulsatile arterial signal from the patient during metronomic deep breathing;
measuring, using a second sensor integrated into the mobile device, a respiratory signal from the patient during metronomic deep breathing;
receiving, by a processor of the mobile device, the pulsatile arterial signal from the first sensor and the respiratory signal from the second sensor;
determining, by the processor of the mobile device, magnitude of the amplitude variation of the pulsatile arterial signal and magnitude of the respiratory signal; and
computing, by the processor of the mobile device, a congestion prediction index based on the magnitude of the amplitude variation of the pulsatile arterial signal and the magnitude of the respiratory signal.

2. The method of claim 1 further comprises guiding the patient in performing metronomic deep breathing using cues issued by the mobile device.

3. The method of claim 2 wherein the cues issued by the mobile device are one of an auditory cue or a visual cue that are configured to indicate a time to initiate each breath during metronomic deep breathing.

4. The method of claim 1 wherein the first sensor is a photoplethysmograph sensor.

5. The method of claim 1 wherein the second sensor is one of an accelerometer or a camera or ECG electrodes.

6. The method of claim 1 wherein determining the magnitude of the amplitude variation of the pulsatile arterial signal further comprises:
determining a difference between a maximum peak-to-peak amplitude of the pulsatile arterial signal and a minimum peak-to-peak amplitude of the pulsatile arterial signal over a respiratory cycle;
determining a mean value based on the maximum peak-to-peak amplitude of the pulsatile arterial signal and the minimum peak-to-peak amplitude of the pulsatile arterial signal; and
dividing the difference by the mean value.

7. The method of claim 1 wherein the magnitude of the respiratory signal is an average peak-to-peak amplitude of the respiratory signal.

8. The method of claim 1, wherein the congestion prediction index is the magnitude of the amplitude variation of the pulsatile arterial signal divided by the magnitude of the respiratory signal.

9. The method of claim 1 further comprises comparing the congestion prediction index or changes in the congestion prediction index over time to a threshold and generating an alert in response the congestion prediction index being less than the threshold.

10. The method of claim 1 further comprises calibrating the respiratory signal for the patient by breathing into a bag of known volume or using an independent respiratory measurement.

11. A method for predicting an onset of pulmonary congestion symptoms in a patient, comprising:
measuring, by a sensor, a pulsatile arterial signal from a patient with a persistent arrhythmia;
receiving, by a processor of a computing device, the pulsatile arterial signal from the sensor;
detecting, by the processor of the computing device, amplitude in the pulsatile arterial signal and length of the beats in the pulsatile arterial signal; and
computing, by the processor of the computing device, a congestion prediction index based on the variation in the detected amplitudes and beat lengths.

12. The method of claim 11 wherein the persistent arrhythmia is atrial fibrillation.

13. The method of claim 11 wherein the sensor is a photoplethysmograph sensor.

14. The method of claim 11 wherein an ECG signal from a second sensor is analyzed to compute the congestion prediction index.

15. The method of claim 11 wherein the congestion prediction index is computed as the slope of the line that relates the peak-to-peak amplitudes of the pulsatile arterial signal, normalized by mean peak-to-peak amplitude of the pulsatile arterial signal, to previous beat lengths of the pulsatile arterial signal.

16. The method of claim 15 wherein the previous beat lengths are normalized by mean beat length.

17. The method of claim 11 wherein the congestion prediction index is computed as standard deviation of the peak-to-peak amplitudes of the pulsatile arterial signal, normalized by the mean peak-to-peak amplitude of the pulsatile arterial signal, divided by the standard deviation of the beat lengths of the pulsatile arterial signal, normalized by mean beat length.

18. The method of claim 11 further comprises comparing the congestion prediction index or changes in the congestion prediction index over time to a threshold and generating an alert in response the congestion prediction index being less than the threshold.

19. The method of claim 11 further comprises excluding short beats or long beats from the computation of the congestion prediction index.

20. A method for predicting an onset of pulmonary congestion symptoms in a patient, comprising:
measuring, by a sensor integrated into a wearable computing device, a pulsatile arterial signal from the patient;
receiving, by a computer processor integrated into the wearable computing device, the pulsatile arterial signal from the sensor;
analyzing, by the computer processor, the pulsatile arterial signal to detect an occurrence of an arrhythmia; and
computing, by the computer processor, a congestion prediction index based on the amplitude variation of the pulsatile arterial signal during the arrhythmia.

22. The method of claim 20 wherein the arrhythmia is paroxysmal atrial fibrillation or a premature beat.

23. The method of claim 20 wherein the sensor is a photoplethysmograph sensor.

24. The method of claim 20 further comprises detecting a period of reduced motion by the patient using an accelerometer integrated into the wearable computing device and analyzing the pulsatile arterial signal during the period of reduced motion.

25. The method of claim 20 further comprises detecting a period of non-vasoconstriction using a temperature sensor integrated into the wearable computing device and analyzing the pulsatile arterial signal during the period of non-vasoconstriction.

26. The method of claim 20 wherein an ECG sensor is integrated into the wearable computing device to facilitate arrhythmia detection and congestion prediction index computation.

27. The method of claim 20 further comprises detecting an occurrence of an arrhythmia based on variation in the beat length of the pulsatile arterial signal.

28. The method of claim 20 wherein the congestion prediction index is computed as slope of a line that relates peak-to-peak amplitudes of the pulsatile arterial signal, normalized by mean peak-to-peak amplitude of the pulsatile arterial signal, to previous beat lengths of the pulsatile arterial signal, normalized by mean beat length.

29. The method of claim 20 wherein the pulsatile arterial signal is analyzed to detect premature beat patterns that are similar in beat lengths and premature beat amplitude, and to compute the congestion prediction index as peak-to-peak amplitude of the beat following a longest beat normalized by peak-to-peak amplitude of a normal beat.

30. The method of claim 20 wherein the congestion prediction index is computed from peak-to-peak amplitude of a beat following a longest beat normalized by peak-to-peak amplitude of a normal beat and from at least one of the premature and subsequent beat lengths and premature beat amplitude.
